(19) United States
(12) Patent Application Publication
(10) Pub. No.: US 2026/0142015 A1
(43) Pub. Date: May 21, 2026

(54) ELECTRONIC DEVICE, USER TERMINAL AND METHOD FOR GUIDING GOAL SETTING AND GOAL ACHIEVEMENT BASED ON BODY INDICATORS

(71) Applicant: InBodyHealthcare Co., Ltd., Seoul (KR)

(72) Inventors: Ki Chul CHA, Seoul (KR); Won Young JANG, Gyeonggi-do (KR); Soo Cheol LIM, Seoul (KR); Ji Hye HWANG, Seoul (KR)

(21) Appl. No.: 19/313,622

(22) Filed: Aug. 28, 2025

(30) Foreign Application Priority Data
Aug. 29, 2024 (KR) 10-2024-0116950
Feb. 18, 2025 (KR) 10-2025-0020878
Aug. 26, 2025 (KR) 10-2025-0119532

(51) Int. Cl. G16H 20/30

(52) U.S. Cl. CPC G16H 20/30

(57) ABSTRACT

An electronic device, a user terminal, and an operating method for guiding goal setting and goal achievement based on body indicators are disclosed. The operating method of the electronic device may include selecting, from among a plurality of clusters based on body indicators, a target cluster that a target user aims for, based on a user input, determining, based on time series data of body indicators pre-obtained from a plurality of users, a target route that guides the target user from a starting cluster including a body indicator of the target user to the target cluster, determining, based on the time series data, a difficulty level and expected time to be taken for the body indicator of the target user to move from the starting cluster to a most adjacent cluster on the target route, and providing the target route, the time, and the difficulty level to the target user.

### TECHNICAL FIELD

An electronic device, a user terminal, and an operating method for guiding goal setting and goal achievement based on body indicators are disclosed.

### BACKGROUND

Devices for measuring body composition using different electrical resistances of fat and muscle are known in the prior art. A body composition measuring device may measure bioimpedance by placing electrodes into contact with an area to be measured to determine body composition. Body composition may be used as an important indicator for evaluating the health status and fitness level of a subject. By analyzing various body components that cannot be determined solely based on weight and height, body composition may be used as an indicator for evaluating the health status, nutritional status, exercise effectiveness, and the like of an individual.

A bioelectrical impedance analysis (BIA) method, which is one of the methods of measuring body composition, passes a small electrical current through the body using electrodes. The BIA method measures impedance based on electrical characteristics of each body part obtained by passing a small electrical current through the body and estimates body composition based on the impedance.

The above description is information the inventor(s) acquired during the course of conceiving the present disclosure, or already possessed at the time, and is not necessarily art publicly known before the present application was filed.

### SUMMARY

### Technical Goals

The present disclosure provides an electronic device and a user terminal that set and provide, utilizing accumulated data, a final route with the highest possibility of achieving a user's goal and that provide a difficulty level and time required to achieve the user's goal.

The present disclosure provides an electronic device and a user terminal that may provide, utilizing accumulated data, specific directions for achieving a goal regarding a degree of increase or decrease in weight and muscle mass and time required to achieve the goal, based on the user's body indicators.

### Technical Solutions

According to an embodiment, an operating method of an electronic device may include selecting, from among a plurality of clusters based on body indicators, a target cluster that a target user aims for, based on a user input, determining, based on time series data of body indicators pre-obtained from a plurality of users, a target route that guides the target user from a starting cluster comprising a body indicator of the target user to the target cluster, determining, based on the time series data, a difficulty level and expected time to be taken for the body indicator of the target user to move from the starting cluster to a most adjacent cluster on the target route, and providing the target route, the time, and the difficulty level to the target user.

The determining of the target route may include determining, from among a plurality of paths, a plurality of candidate paths based on the starting cluster and the target cluster, wherein the plurality of paths is predetermined based on an index representing position information of the body indicators, and determining a target path among the plurality of candidate paths and determining the target route based on the target path.

The determining of the plurality of candidate paths may include determining a plurality of candidate paths starting from an index representing the body indicator of the target user and ending at any index included in the target cluster.

The determining of the expected time and the difficulty level may include extracting, from the time series data, data with an index changed compared to previous data, wherein the index represents statistical position information of the body indicator, determining movement rate information representing a rate of movement from each index to each of adjacent indices, based on the extracted data, and determining the difficulty level based on the movement rate information.

The determining of the difficulty level based on the movement rate information may include generating, based on the movement rate information, a histogram with a rate and a number of pieces of data included in the movement rate information as axes, determining a cumulative distribution function based on the histogram, and determining the difficulty level based on the cumulative distribution function.

The determining of the expected time and the difficulty level may include extracting, from the time series data, data with an index changed compared to previous data, wherein the index represents statistical position information of the body indicator, determining, based on the extracted data, time information representing time taken per unit distance consumed in moving from each index to each of adjacent indices, and determining the expected time based on the time information.

The determining of the time information may include determining the time information representing the time information representing the time taken per unit distance by dividing time consumed in moving from each index to each of the adjacent indices in a coordinate space, which uses the body indicators as coordinates, by a movement distance of the index.

The body indicators may include a body mass index (BMI) and a percentage body fat (PBF).

The selecting of the target cluster may include selecting, based on the user input, any one of top two or more clusters with the highest scores based on the body indicators as the target cluster.

The selecting of the target cluster may include selecting, based on the user input, any one of clusters with scores based on the body indicators greater than or equal to an average as the target cluster.

The selecting of the target cluster may include selecting, based on the user input, any one of clusters determined based on a period set by the target user as the target cluster.

According to an embodiment, an operating method of a user terminal may include obtaining a user input from a target user to select, from among a plurality of clusters based on body indicators, a target cluster that the target user aims for, displaying, in response to obtaining the user input, a target route that provides a guide to reach the target cluster from a starting cluster comprising a body indicator of the target user, and displaying a difficulty level and expected time to be taken for the body indicator of the target user to move from the starting cluster to a most adjacent cluster on the target route, wherein an electronic device communicating with the user terminal may be configured to, in response to receiving the user input from the user terminal, determine the target route, the time, and the difficulty level based on time series data of body indicators pre-obtained from a plurality of users, and provide the target route, the expected time, and the difficulty level to the user terminal.

According to an embodiment, an electronic device may include at least one processor comprising processing circuitry and memory storing instructions that may, when executed by the at least one processor individually or collectively, cause the electronic device to select, from among a plurality of clusters based on body indicators, a target cluster that a target user aims for, based on a user input, determine, based on time series data of body indicators pre-obtained from a plurality of users, a target route that guides the target user from a starting cluster comprising a body indicator of the target user to the target cluster, determine, based on the time series data, a difficulty level and expected time to be taken for the body indicator of the target user to move from the starting cluster to a most adjacent cluster on the target route, and provide the target route, the time, and the difficulty level to the target user.

When executed by the at least one processor individually or collectively, the instructions may cause the electronic device to determine, from among a plurality of paths, a plurality of candidate paths based on the starting cluster and the target cluster, wherein the plurality of paths is predetermined based on an index representing position information of the body indicators, and determine a target path among the plurality of candidate paths and determine the target route based on the target path.

When executed by the at least one processor individually or collectively, the instructions may cause the electronic device to determine a plurality of candidate paths starting from an index representing the body indicator of the target user and ending at any index included in the target cluster.

When executed by the at least one processor individually or collectively, the instructions may cause the electronic device to extract, from the time series data, data with an index changed compared to previous data, wherein the index represents statistical position information of the body indicator, determine movement rate information representing a rate of movement from each index to each of adjacent indices, based on the extracted data, and determine the difficulty level based on the movement rate information.

When executed by the at least one processor individually or collectively, the instructions may cause the electronic device to generate, based on the movement rate information, a histogram with a rate and a number of pieces of data included in the movement rate information as axes, determine a cumulative distribution function based on the histogram, and determine the difficulty level based on the cumulative distribution function.

When executed by the at least one processor individually or collectively, the instructions may cause the electronic device to extract, from the time series data, data with an index changed compared to previous data, wherein the index represents statistical position information of the body indicator, determine, based on the extracted data, time information representing time taken per unit distance consumed in moving from each index to each of adjacent indices, and determine the expected time based on the time information.

When executed by the at least one processor individually or collectively, the instructions may cause the electronic device to determine the time information representing the time information representing the time taken per unit distance by dividing time consumed in moving from each index to each of the adjacent indices in a coordinate space, which uses the body indicators as coordinates, by a movement distance of the index.

The body indicators may include a BMI and a PBF.

### Effects

According to an embodiment of the present disclosure, an electronic device and a user terminal may, based on accumulated data, set and provide a route with the highest possibility of achieving a user's goal and may provide a user with a difficulty level and time required to achieve the user's goal.

According to an embodiment of the present disclosure, an electronic device and a user terminal may, based on accumulated data, provide a user with specific directions for achieving a goal regarding a level of increase or decrease in weight, a level of increase or decrease in muscle mass, and time required in the user's current state to achieve a goal.

### BRIEF DESCRIPTION OF THE DRAWINGS

FIG. 1 is a diagram illustrating a body indicator measuring device, a user terminal, and an electronic device, according to an embodiment.

FIG. 2 is a diagram illustrating a cluster according to an embodiment.

FIG. 3 is a diagram illustrating an operation of an electronic device, according to an embodiment.

FIG. 4 is a flowchart illustrating a screen displayed on a user terminal, according to an embodiment.

FIGS. 5 and 6 are diagrams illustrating a selection of a target cluster, according to an embodiment.

FIG. 7 is a diagram illustrating a determination of a target route, according to an embodiment.

FIG. 8 is a flowchart illustrating an operating method of an electronic device for determining a difficulty level and expected time, according to an embodiment.

FIG. 9 is a diagram illustrating time series data according to an embodiment.

FIG. 10 is a diagram illustrating a movement direction according to an embodiment.

FIG. 11 is a diagram illustrating interpolated time series data according to an embodiment.

FIG. 12 is a diagram illustrating movement rate information according to an embodiment.

FIG. 13 is a diagram illustrating a histogram and cumulative distribution function based on movement rate information, according to an embodiment.

FIGS. 14 and 15 are diagrams illustrating a determination of expected time, according to an embodiment.

FIG. 16 is a flowchart illustrating an operation of a user terminal, according to an embodiment.

FIG. 17 is a diagram illustrating an electronic device according to an embodiment.

### DETAILED DESCRIPTION

Hereinafter, embodiments are described in detail with reference to the accompanying drawings. The scope of the right, however, should not be construed as limited to the embodiments set forth herein. In the drawings, like reference numerals are used for like elements.

Various modifications may be made to the embodiments. Here, the embodiments are not construed as limited to the disclosure and should be understood to include all changes, equivalents, and replacements within the idea and the technical scope of the disclosure.

Although terms such as “first” or “second” are used to explain various components, these terms should be used only to distinguish one component from another component. For example, a first component may be referred to as a second component, and similarly, the second component may be referred to as the first component.

The terminology used herein is for the purpose of describing particular embodiments only and is not intended to limit the embodiments. The singular forms are intended to include the plural forms as well, unless the context clearly indicates otherwise. As used herein, each of phrases such as “A or B,” “at least one of A and B,” “at least one of A or B,” “A, B, or C,” “at least one of A, B, and C,” and “at least one of A, B, or C” may include any one of the items listed in the corresponding one of the phrases or all possible combinations thereof. It should be further understood that the terms “comprises/comprising” and/or “includes/including,” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical or scientific terms used herein have the same meaning as those commonly understood by one of ordinary skill in the art to which the embodiments belong. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

When describing the embodiments with reference to the accompanying drawings, like reference numerals refer to like components and a repeated description related thereto is omitted. In the description of embodiments, detailed description of well-known related structures or functions is omitted when it is deemed that such description may cause ambiguous interpretation of the present disclosure.

Hereinafter, embodiments are described in detail with reference to the accompanying drawings.

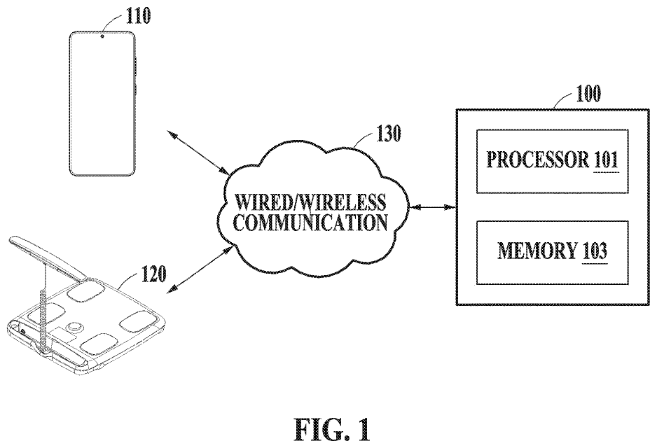

FIG. 1 is a diagram illustrating a body indicator measuring device, a user terminal, and an electronic device, according to an embodiment.

Referring to FIG. 1, an electronic device 100, a user terminal 110, and a body indicator measuring device 120 are illustrated. The electronic device 100 may be a device of a service provider that provides services described in the present disclosure. The electronic device 100 may be, for example, a server, but is not limited thereto.

The electronic device 100, the user terminal 110, and the body indicator measuring device 120 may communicate with each other via wired/wireless communication 130. The wired/wireless communication 130 may include bidirectional communication and/or one-way communication. The wired/wireless communication 130 may include short-range communication (e.g., wireless fidelity (Wi-Fi), Bluetooth, ultra-wideband (UWB), near-field communication (NFC), infrared data association (IrDA), or radio frequency identification (RFID)) and/or medium-to long-range communication (e.g., cellular communication or satellite communication).

The body indicator measuring device 120 may measure a body indicator of a user. The body indicator measuring device 120 may transmit the body indicator of the user to the user terminal 110 and/or the electronic device 100 via the wired/wireless communication 130. The body indicator measuring device 120 may receive information such as height, weight, and age of the user in advance as an input for measuring the body indicator. The body indicator may include body composition and indicators related to a body other than body composition. Body composition may include, for example, percent body fat (PBF), lean body mass (LBM), skeletal muscle mass (SMM), muscle mass, visceral fat mass, total body water (TBW), protein mass, mineral mass, fat mass, extracellular water ratio, abdominal fat rate, and segmental muscle mass, but is not limited thereto. The indicators related to a body may include body mass index (BMI), score, and basal metabolic rate (BMR), but are not limited thereto.

The user terminal 110 may provide the user with the received body indicator of the user. The user terminal 110 may provide the user with changing trends of the body indicator of the user. The user terminal 110 may include various computing devices, such as a mobile phone, a smartphone, a tablet, an electronic book reader, a laptop, a personal computer (PC), a desktop, or a workstation, and various wearable devices, such as a smartwatch, smart glasses, or a head-mounted display (HMD).

The electronic device 100 may include a processor 101 and a memory 103. However, this is only an example and should not be construed as limiting other embodiments. For example, it is obvious to one of ordinary skill in the art that the electronic device may further include general-purpose components.

The processor 101 may perform overall functions for controlling the electronic device 100. The processor 101 may generally control the electronic device 100 by executing programs and/or instructions stored in the memory 103. The processor 101 may be implemented as, but is not limited to, a central processing unit (CPU), a graphics processing unit (GPU), an application processor (AP), and the like that are included in the electronic device 100. The number of the processor 101 may be one or more. For example, the processor 101 may have a multi-core processor structure, such as a dual core, quad core, or hexa core. The processor 101 may control operations of the electronic device 100 by executing instructions stored in the memory 103. For example, the processor 101 may correspond to a plurality of processors that collectively perform a plurality of operations by dividing the operations among the processors.

The memory 103 may be hardware for storing data that has been processed or that is to be processed in the electronic device 100. Furthermore, the memory 103 may store applications or drivers to be driven by the electronic device 100. In addition, the memory 103 may include instructions. The memory 103 may include one or more memories. The instructions stored in the memory 103 may be stored in a single memory. The instructions stored in the memory 103 may be divided and stored in a plurality of memories. The memory 103 may include volatile memory, such as dynamic random-access memory (DRAM), and/or nonvolatile memory.

The electronic device 100 may store the received body indicator of the user to provide services. The electronic device 100 may store the body indicator of the user and/or body indicators of other users. The electronic device 100 may accumulate body indicators received from the user or other users. The electronic device 100 may manage the accumulated body indicators as time series data for each user. The electronic device 100 may provide services to the user and/or other users based on accumulated data. For example, the electronic device 100 may provide the user or other users with services of guidance on goal setting and goal achievement based on the body indicators. To provide the services of guidance on goal setting and goal achievement, the electronic device 100 may perform clustering on the accumulated data to generate a plurality of clusters. The electronic device 100 may provide services based on the plurality of clusters. The clusters are described below.

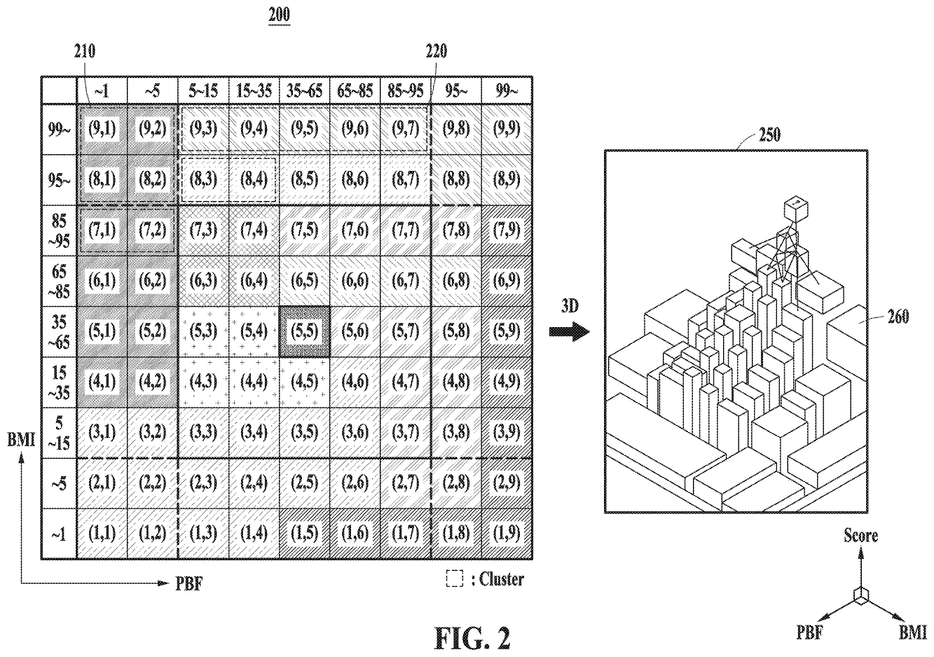

FIG. 2 is a diagram illustrating a cluster according to an embodiment.

Referring to FIG. 2, a cluster map 200 is illustrated. An electronic device may accumulate body indicators measured from users. The electronic device may classify types (e.g., clusters) of the users based on two or more of the accumulated body indicators.

For ease of description, the present disclosure focuses on a case in which user types are classified based on BMI and PBF. However, it is obvious to one of ordinary skill in the art that the description of the present disclosure may equally apply to cases of classifying user types based on body indicators (e.g., SMM or LBM) other than BMI and PBF.

Since only a portion (e.g., a cluster 210 and a cluster 220) of the clusters is shown in the present disclosure, it should be understood that more clusters exist on the cluster map 200. For ease of description, the present disclosure describes a case with nine percentile segments. However, it is obvious to one of ordinary skill in the art that the description below may equally apply to cases with fewer or more percentile segments than nine. A method of generating a plurality of clusters is described below.

The electronic device may display users on a two-dimensional (2D) space with BMI (e.g., y-axis) and PBF (e.g., x-axis) as axes. The electronic device may display a plurality of users on the 2D space with BMI and PBF as axes, using BMI and PBF, which may be among accumulated body indicators. The electronic device may divide the plurality of users into a plurality of percentile segments for BMI and PBF, respectively. A percentile segment may show a proportion of the plurality of users in the corresponding segment. The plurality of percentile segments may include, but are not limited to, less than 1%, 1% or more but less than 5%, 5% or more but less than 15%, 15% or more but less than 35%, 35% or more but less than 65%, 65% or more but less than 85%, 85% or more but less than 95%, 95% or more but less than 99%, and 99% or more. A percentile segment with a higher percent may indicate a proportion of individuals with a higher BMI or PBF. For example, a BMI of an individual whose BMI falls within the less than 1% percentile may be lower than a BMI of an individual whose BMI falls within the 99% or more percentile. For example, a PBF of an individual whose PBF falls within the less than 1% percentile may be lower than a PBF of an individual whose PBF falls within the 99% or more percentile.

The electronic device may divide a 2D space with BMI and PBF as axes into 9 percentile segments, and thus, 81 (e.g., 9×9) cells may be defined. Each cell may have an index. The index of each cell may represent statistical position information of a body indicator. The index may be expressed as (BMI_index, PBF_index). For example, individuals in a cell with index (1, 1) may represent individuals whose BMI and PBF are both within the top 1% of all users. For example, individuals in a cell with index (3, 6) may represent individuals, among all users, whose BMI is 5% or more but less than 15% and whose PBF is 65% or more but less than 85%.

An index may represent statistical position information and may thus be determined differently by gender. For example, men and women included in the same index may have different PBFs and/or BMIs. For example, a BMI of a male included in index (4, 3) may be 15, while a BMI of a woman included in index (4, 3) may be 25. An index may represent statistical position information and may thus be determined differently by age. For example, a BMI of a man in his 20s included in index (4, 3) may be 15, while a BMI of a man in his 50s included in index (4, 3) may be 22. Since an index may be determined differently by gender and/or by age, individuals corresponding to the same index may have different body indicators depending on gender and/or age.

The electronic device may perform clustering based on body indicators of individuals included in each index. Individuals included in adjacent indices may have common clinical characteristics. The electronic device may cluster one or more adjacent indices to determine a plurality of clusters and generate a cluster map 200. The electronic device may group indices with common or similar clinical characteristics to determine the plurality of clusters and generate the cluster map 200. A cluster may include one or more indices. For example, a cluster 210 may include (9, 1), (8, 1), (9, 2), and (8, 2). For example, a cluster 220 may include (9, 3), (9, 4), (9, 5), (9, 6), and (9, 7). Each cluster may have a nickname. For example, the cluster 210 may have a nickname related to a characteristic of the corresponding cluster, such as “Go, the world's strongest man.”

The electronic device may represent the plurality of clusters in three dimensions. For example, the electronic device may represent the plurality of clusters as a three-dimensional (3D) cluster map 250. The electronic device may represent the plurality of clusters in a 3D space with PBF, BMI, and score as axes. The electronic device may determine a score for a cluster based on body indicators. For example, the electronic device may determine the score by inputting a representative value among the body indicators included in a cluster to a predetermined formula. A cluster may include body indicators that users consider ideal as the score increases. The cluster 210 may be displayed as a cluster 260 in the 3D cluster map 250.

Hereinafter, a method of guiding goal setting and goal achievement based on the plurality of clusters is described.

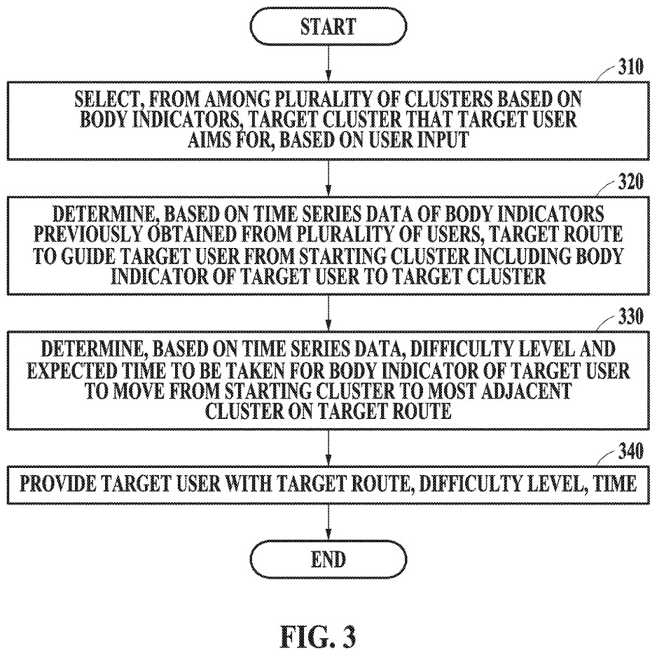

FIG. 3 is a diagram illustrating an operation of an electronic device, according to an embodiment.

In the following embodiments, operations may be performed sequentially but not necessarily. For example, the order of the operations may change, and at least two of the operations may be performed in parallel. In addition, some of the operations may be omitted depending on the embodiment. Operations 310 to 340 may be performed by at least one component (e.g., a processor) of the electronic device.

According to an embodiment, instructions stored in memory by at least one processor may be executed individually or collectively and may cause the electronic device to perform the following operations.

In operation 310, the electronic device may select, from among a plurality of clusters based on body indicators, a target cluster that a target user aims for, based on a user input.

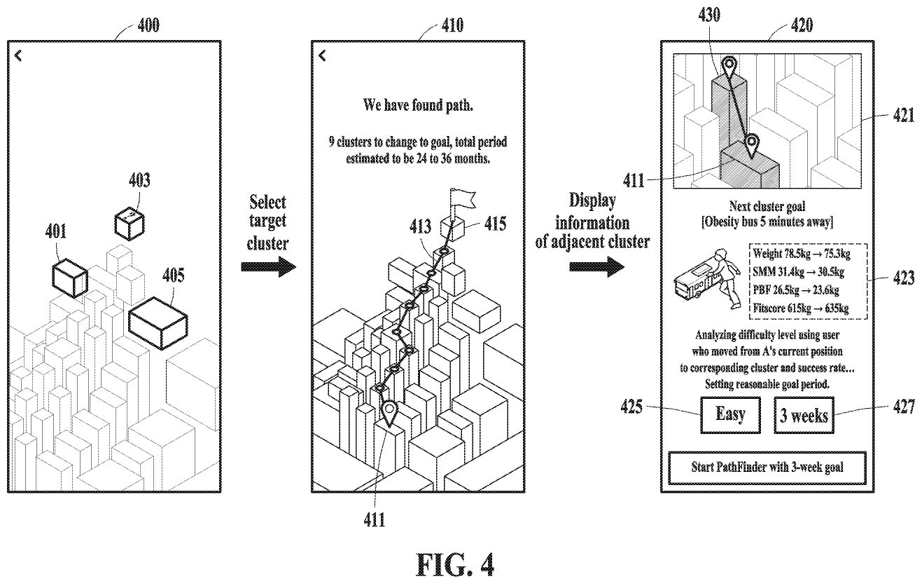

The target user may be a user that requests guidance on goal setting and goal achievement based on body indicators. The electronic device may receive a user input indicating a selection of any one of top two or more clusters with highest scores. The electronic device may select the target cluster from among the top two or more clusters with the highest scores based on the user input. The selection of the target cluster is further described below with reference to FIGS. 4 to 6.

In operation 320, the electronic device may determine, based on time series data of body indicators previously obtained from a plurality of users, a target route to guide the target user from a starting cluster including a body indicator of the target user to the target cluster.

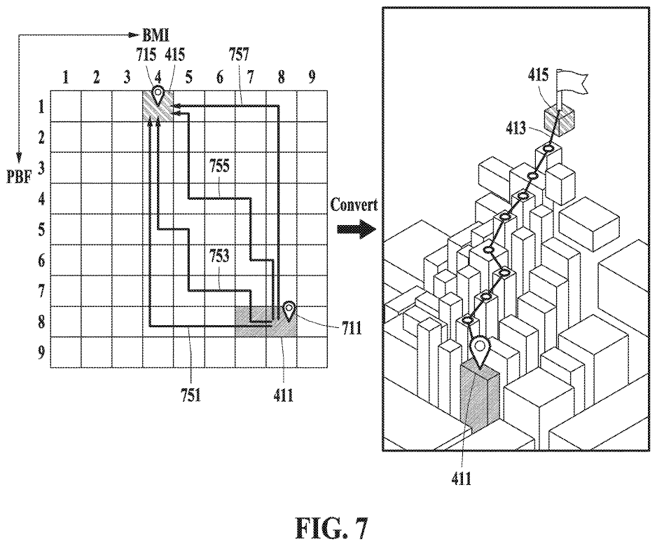

A method of determining the target route is described below with reference to FIG. 7.

In operation 330, the electronic device may determine, based on the time series data, a difficulty level and expected time to be taken for the body indicator of the target user to move from the starting cluster to a most adjacent cluster on the target route.

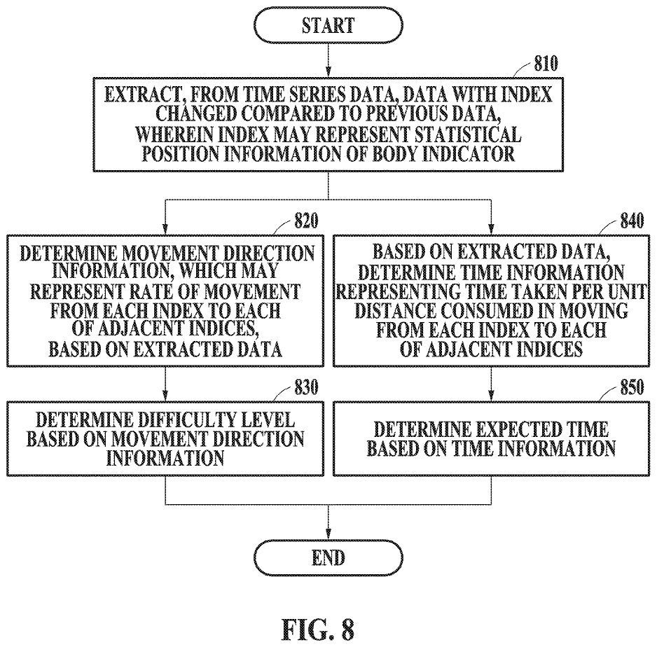

The electronic device may extract, from the time series data, data with an index changed compared to previous data, wherein the index may represent statistical position information of a body indicator. The electronic device may determine the difficulty level and the expected time, based on the extracted data. A method of determining the difficulty level and the expected time is described below with reference to FIGS. 8 to 15.

In operation 340, the electronic device may provide the target user with the target route, the difficulty level, and the time.

The electronic device may transmit the target route to a user terminal. The user terminal may display the received target route. The electronic device may transmit, to the user terminal, the difficulty level and the expected time to be taken to move from the starting cluster to the most adjacent cluster on the target route. The user terminal may display the difficulty level and the expected time to be taken to move from the starting cluster to the most adjacent cluster on the target route.

The electronic device may determine a difficulty level and expected time to be taken for the target user to move along the target route to a next cluster on the target route until reaching the target cluster. When a change in a cluster is identified, the electronic device may determine a difficulty level and expected time to be taken to move from the changed cluster to a most adjacent cluster on the target route. When a change in a cluster is identified, the electronic device may determine and provide the difficulty level and the expected time to be taken to move from the changed cluster to the most adjacent cluster on the target route, until the target user reaches the target cluster.

The electronic device may provide a competitor option, which may allow comparing a goal achievement progress of the target user with a competitor. The electronic device may set a competitor from among a plurality of users included in the same cluster, who has selected the same goal as the target user. For example, the electronic device may automatically select, as competitors, top 3 users having the greatest change in biomarkers at a timepoint when a predetermined period (e.g., 5 days) passed after setting a goal. For example, the electronic device may set, as competitors, users directly designated by the target user.

The electronic device may obtain time series data on changing trends of body indicators and/or changing trends of scores of the competitors and display them along with changing trends of the target user. For example, the electronic device may sort goal setting dates of the target user and the competitors by a same reference date and display the changing trends of the target user and the competitors together.

When the competitors drop out or when the target user provides an input to change or delete the competitors, the electronic device may update a competitor configuration accordingly.

The electronic device may additionally provide information to determine how many other users have designated the target user as a competitor.

Hereinafter, a screen displayed on the user terminal is described.

FIG. 4 is a flowchart illustrating a screen displayed on a user terminal, according to an embodiment.

Referring to FIG. 4, a screen of the user terminal is illustrated, which may provide a selection of a target cluster. The user terminal may be a terminal of a target user. The user terminal may execute an application that provides a body indicator of the target user, obtained from a body indicator measuring device. The user terminal may provide, via the application, goal setting and goal achievement based on the body indicator. The user terminal may display a screen 400 when receiving a user input requesting the goal setting and the goal achievement based on the body indicator.

The user terminal may obtain a user input from the target user to select, from among a plurality of clusters based on body indicators, a target cluster 415 that the target user aims for.

Referring to the screen 400, the user terminal may provide the target user with a selection of top two or more clusters with highest scores based on body indicators. For example, the user terminal may provide the target user with a selection of any one among a cluster 401, a cluster 403, and a cluster 405, which may be top three clusters with the highest scores. The user terminal may obtain a user input selecting any one cluster as the target cluster 415. The user terminal may transmit the user input to an electronic device. The electronic device may, based on the user input, select any one of the top two or more clusters with the highest scores based on the body indicators as the target cluster 415. When the target cluster 415 is selected, the electronic device may determine a target route 413 and transmit it to the user terminal. The user terminal may receive the target route 413 from the electronic device in response to transmitting the user input to the electronic device.

On a screen 410, the user terminal may display the target route 413. The user terminal may display the target route 413 that provides a guide to reach the target cluster 415 from a starting cluster 411 including the body indicator of the target user. The target route 413 may include the starting cluster 411 and the target cluster 415. The target route 413 may include a transit cluster. For example, the target route 413 may include 8 transit clusters. A transit cluster may be a cluster passed through while traveling from the starting cluster 411 to the target cluster 415 along the target route 413.

Referring to the screen 410, the user terminal may display an approximate time expected to be taken to reach the target cluster 415 from the starting cluster 411. For example, the user terminal may display “A total required time is estimated to be 24 to 36 months.”

Referring to a screen 420, an animation 421 may be provided in which the body indicator of the target user moves from the starting cluster 411 to a cluster (e.g., a next cluster 430) that is most adjacent to the starting cluster on the target route. The user terminal may display detailed information for the body indicator of the target user to move from the starting cluster 411 to the cluster (e.g., the next cluster 430 or a transit cluster) that is most adjacent to the starting cluster 411 on the target route 413.

Referring to the screen 420, the user terminal may display change information 423 of at least a portion of the body indicator that is required to move from the starting cluster 411 to the next cluster 430. Referring to the screen 420, the user terminal may display a difficulty level 425 and expected time 427 to be taken for the body indicator of the target user to move from the starting cluster 411 to the most adjacent cluster (e.g., a next cluster) on the target route 413. The user terminal may receive, from the electronic device, the difficulty level 425 and the expected time 427 to be taken for the body indicator of the target user to move from the starting cluster 411 to the most adjacent cluster on the target route 413 to display.

Hereinafter, a method of selecting the target cluster 415 is further described.

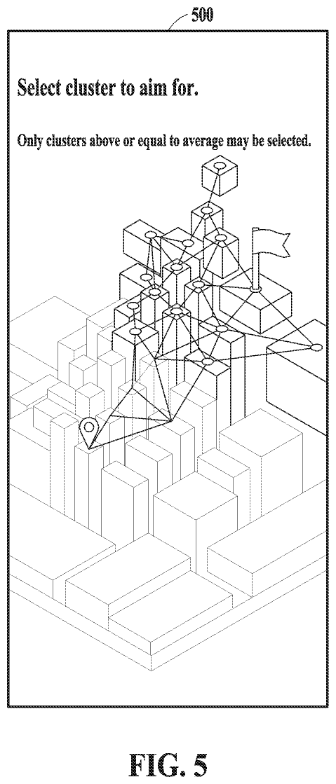

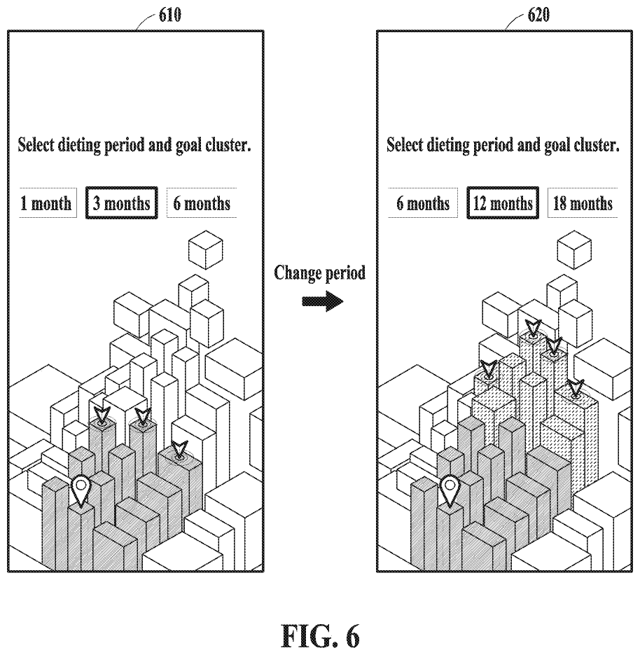

FIGS. 5 and 6 are diagrams illustrating a selection of a target cluster, according to an embodiment.

Referring to FIG. 5, a user terminal may display a screen 500 that provides a target user with a selection of any one of clusters with scores based on body indicators greater than or equal to an average. The user terminal may receive a user input that selects, as a target cluster, any one of the clusters with scores greater than or equal to the average. The user terminal may transmit the user input to an electronic device. Based on the user input, the electronic device may select, as the target cluster, any one of the clusters with scores based on body indicators greater than or equal to the average.

Referring to FIG. 6, the user terminal may display a screen 610 that provides the target user with a selection of any one of clusters determined based on a period set by the target user.

Referring to the screen 610, the user terminal may provide to the target user with a selection of any one of clusters expected to be reachable within 3 months, based on the period of 3 months set by the target user. The user terminal may receive a user input for any one of the clusters. The user terminal may transmit the user input to an electronic device. The electronic device may select, as the target cluster, any one of the clusters determined based on the period (e.g., 3 months) set by the target user, based on the user input.

Referring to a screen 620, the user terminal may provide the target user with a selection of any one of clusters expected to be reachable within 12 months, based on the period of 12 months set by the target user. As the period extends, farther clusters with respect to a starting cluster may be provided. The user terminal may receive a user input for any one of the clusters. The user terminal may transmit the user input to the electronic device. The electronic device may select, as the target cluster, any one of the clusters determined based on the period (e.g., 12 months) set by the target user, based on the user input.

Hereinafter, a method of determining a target route is described.

FIG. 7 is a diagram illustrating a determination of a target route, according to an embodiment.

When the target cluster 415 is determined, an electronic device may determine the target route 413 that reaches the target cluster 415 from the starting cluster 411. The starting cluster 411 may be a cluster that includes an index 711 of a latest body indicator obtained from the target user, and the target cluster 415 may be a cluster selected by the target user described above with reference to FIGS. 5 to 7. The target cluster 415 may include one or more indices 715. For ease of description, the starting cluster may be referred to as a cluster in which the target user is currently included.

The electronic device may determine a target path based on indices and convert the target path into the cluster-based target route 413. The electronic device may determine a target path that may reach at least one index 715, included in the target cluster 415, starting from the index 711 of the target user.

The electronic device may include a plurality of predetermined paths. The electronic device may predetermine the plurality of paths based on time series data of a plurality of users. The electronic device may determine, based on the time series data of the plurality of users, changing trends of indices of the plurality of users. The electronic device may predetermine the plurality of paths based on the changing trends of the indices.

The plurality of paths may have different starting indices and/or target indices. For example, one of the plurality of paths may be a path that starts from a first index and reaches a second index, and another one of the plurality of paths may be a path that starts from a third index and reaches a fourth index.

The electronic device may determine, from among the plurality of paths, a plurality of candidate paths 751, 753, 755, and 757 starting from the index 711 of the target user and arriving at any one index 715 included in the target cluster 415.

The electronic device may determine, from among the plurality of paths, the plurality of candidate paths 751, 753, 755, and 757 starting from the index 711 of the target user and arriving at any one index 715 included in the target cluster 415 from within a specified moving range. The specified moving range may be determined as Equation 1 below.

$$Specified moving range = [ \vert {PBF}_{A} - {PBF}_{B} \vert + \vert {BMI}_{A} - {BMI}_{B} \vert , \vert {PBF}_{A} - {PBF}_{B} \vert + \vert {BMI}_{A} - {BMI}_{B} \vert + C ] \qquad Equation 1$$

PBF_{A} may be PBFi included in the index 711 of the target user. BMI_{A} may be BMIi included in the index 711 of the target user. PBF_{B} may be PBFi included in any one index 715 included in the target cluster 415. BMI_{B} may be BMIi included in any one index 715 included in the target cluster 415. C may be a set value set by a user. C may be an integer greater than or equal to 0.

For example, when C equals 0, since the index 711 of the target user may be (2, 7) and any one index 715 included in the target cluster 415 may be (9, 3), the specified moving range may be determined to be 11. The electronic device may extract, from the plurality of paths, the plurality of candidate paths 751, 753, 755, and 757 starting from the index 711 and reaching the index 715 with 11 moves.

For example, when C equals 2, since the index 711 of the target user may be (2, 7) and any one index 715 included in the target cluster 415 may be (9, 3), the specified moving range may be determined to be 11, 13 . The electronic device may extract, from the plurality of paths, the plurality of candidate paths starting from the index 711 and reaching the index 715 with 11 to 13 moves.

The electronic device may determine any one of the plurality of candidate paths 751, 753, 755, and 757 as the target path. The electronic device may determine score increase and decrease trends according to a change in the indices for the plurality of candidate paths 751, 753, 755, and 757. The electronic device may determine the target path based on the score increase and decrease trends. A detailed description on the score of indices is omitted since the description on the score of clusters may apply thereto.

For example, the electronic device may, based on the score increase and decrease trends, determine a path with a least average score increase and decrease as the target path. For example, the electronic device may determine, based on the score increase and decrease trends, a path with a greatest average score increase and decrease as the target path. For example, the electronic device may, based on the score increase and decrease trends, determine a path with the score decreasing in the beginning but increasing in the end as the target path.

The electronic device may convert the target path into a target route. The target path may be a path of an index in two dimensions, and the target route may be a route of a cluster in three dimensions. The target path may be a movement path in units of indices, while the target route may be a route in units of clusters. For example, when the candidate path 753 is determined as the target path, the electronic device may convert the candidate path 753 into the target route 413.

Hereinafter, a method of determining a difficulty level and expected time to be taken for a body indicator of the target user to move from the starting cluster 411 to a most adjacent cluster (e.g., the cluster 710) on the target route 413 is described.

FIG. 8 is a flowchart illustrating an operating method of an electronic device for determining a difficulty level and expected time, according to an embodiment.

In the following embodiments, operations may be performed sequentially but not necessarily. For example, the order of the operations may change, and at least two of the operations may be performed in parallel. In addition, some of the operations may be omitted depending on the embodiment. Operations 810 to 850 may be performed by at least one component (e.g., a processor) of the electronic device.

According to an embodiment, instructions stored in memory by at least one processor may be executed individually or collectively and may cause the electronic device to perform the following operations.

In operation 810, the electronic device may extract, from time series data, data with an index changed compared to previous data, wherein the index may represent statistical position information of a body indicator.

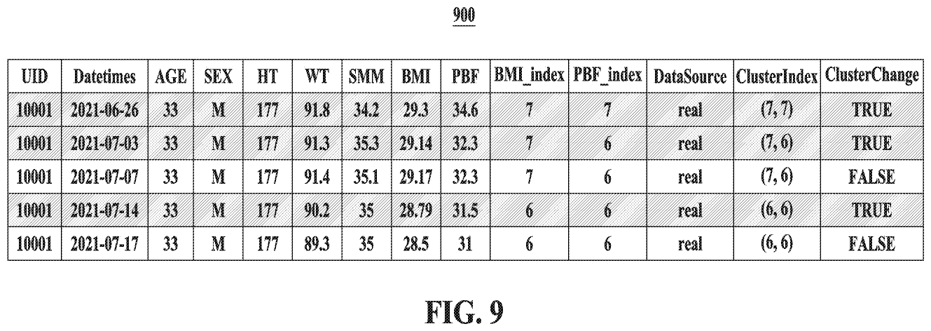

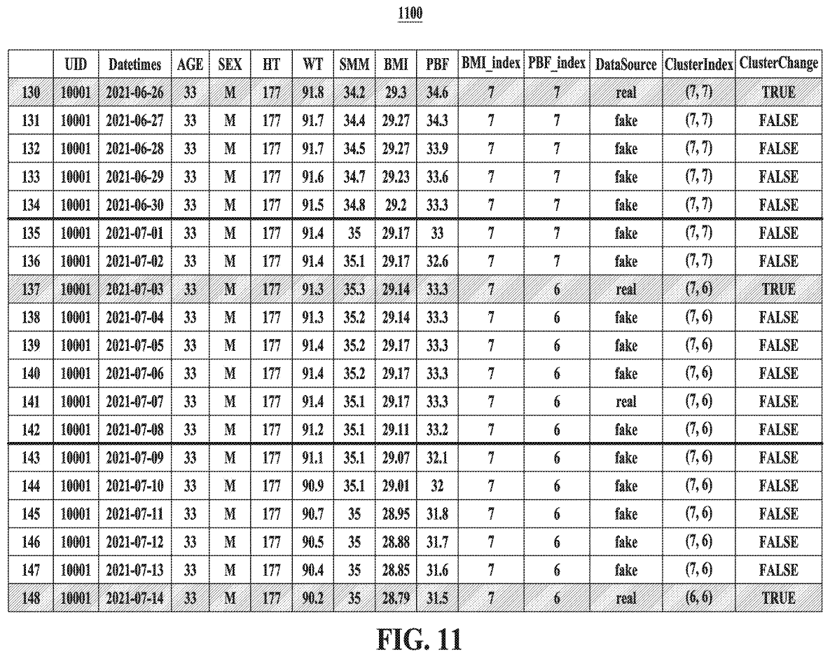

Extraction of data is further described with reference to FIGS. 9 and 11.

In operation 820, the electronic device may determine movement direction information, which may represent a rate of movement from each index to each of adjacent indices, based on the extracted data.

In operation 830, the electronic device may determine a difficulty level based on the movement direction information.

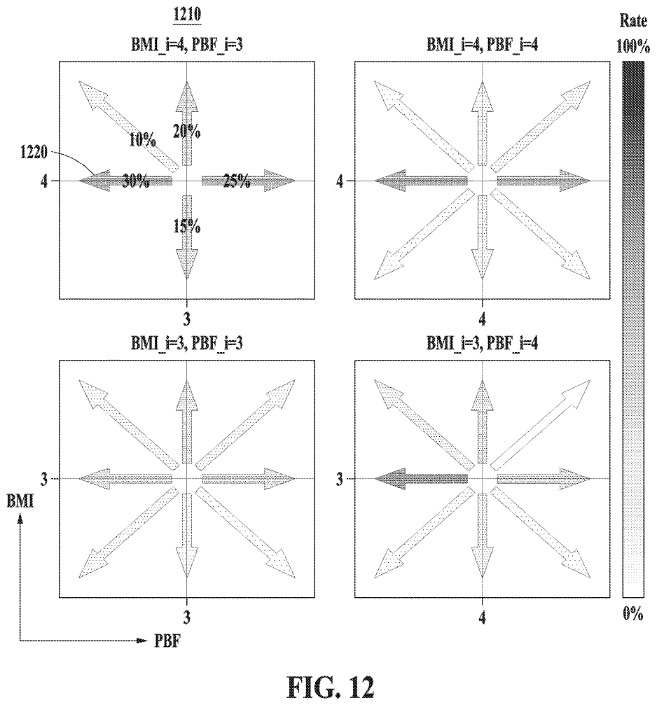

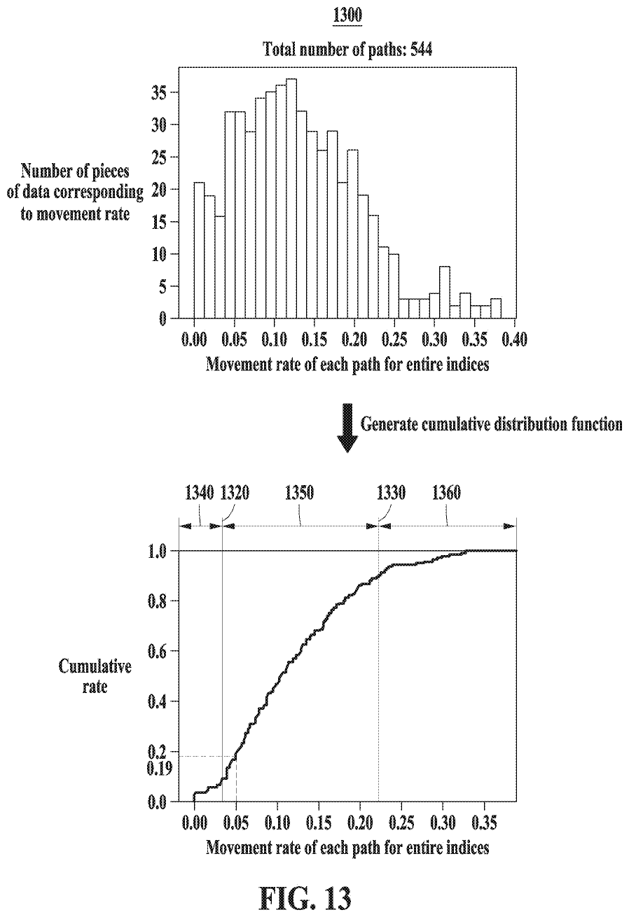

The electronic device may, based on movement rate information, generate a histogram with a rate and a number of pieces of data included in the movement rate information as axes. The electronic device may determine a cumulative distribution function based on the histogram. The electronic device may determine a difficulty level based on the cumulative distribution function. A method of determining the difficulty level is further described with reference to FIGS. 12 and 13.

In operation 840, the electronic device may, based on the extracted data, determine time information representing time taken per unit distance consumed in moving from each index to each of adjacent indices.

The electronic device may determine the time information representing the time taken per unit distance by dividing the time consumed in moving from each index to each of the adjacent indices in a coordinate space, which uses body indicators as coordinates, by a movement distance of the index.

In operation 850, the electronic device may determine expected time based on the time information.

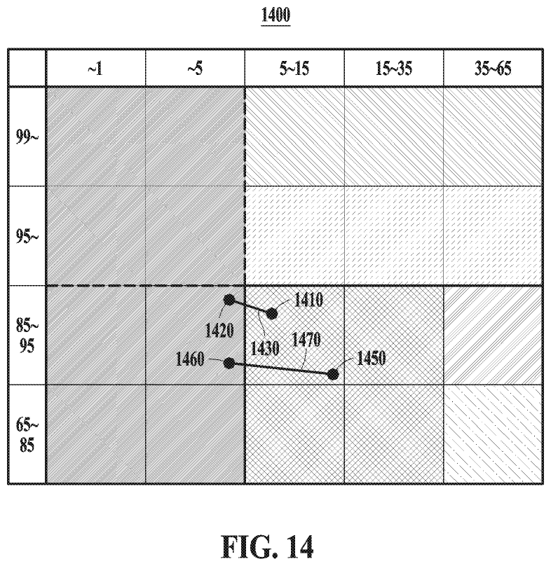

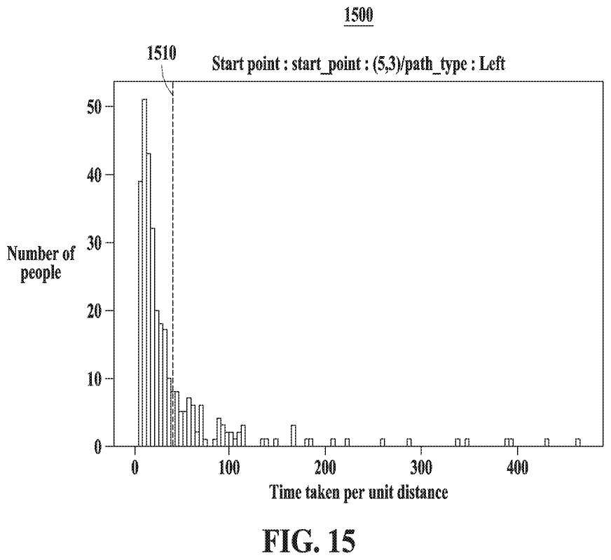

A method of determining time information and determining expected time is further described with reference to FIGS. 14 and 15.

FIG. 9 is a diagram illustrating time series data according to an embodiment. Referring to FIG. 9, a portion of time series data of body indicators pre-obtained from a plurality of users is illustrated. The time series data may be a list of data chronologically measured for each user. For ease of description, the data illustrated in FIG. 9, though being a portion of the time series data of the body indicators pre-obtained from the plurality of users, is referred to as time series data 900 herein.

The time series data 900 may include a plurality of fields. The plurality of fields of the time series data 900 may include, but is not limited to, user identification (UID) (e.g., a user identification number), datetimes (e.g., a date of measurement), age (e.g., age), sex (e.g., sex), height (HT) (e.g., a height), weight (WT) (e.g., a weight), SMM (e.g., skeletal muscle mass), PBF (e.g., percent body fat), BMI (e.g., body mass index), BMI_index, PBF_index, Datasource, ClusterIndex, and ClusterChange. Some of the plurality of fields may be pre-obtained before measurement of body indicators. For example, the age, sex, and HT may be pre-obtained before measurement of body indicators. Some of the plurality of fields may be obtained through measurement of body indicators. For example, the WT, SMM, PBF, BMI, BMI_index, PBF_index, Datasource, ClusterIndex, and ClusterChange may be obtained through measurement of body indicators.

The BMI_index may indicate a position on a cluster map on which a user whose body indicator has been measured is placed with respect to a BMI axis, based on the measured body indicator. The PBF_index may indicate a position on the cluster map on which the user whose body indicator has been measured is placed with respect to a PBF axis, based on the measured body indicator. The Datasource may indicate whether the body indicator is obtained through actual measurement (e.g., real) or obtained through interpolation (e.g., fake). The ClusterIndex may be an index indicating statistical position information of the user whose body indicator has been measured and may be determined based on the BMI_index and the PBF_index. The ClusterIndex may be expressed as (BMI_index, PBF_index). The ClusterChange may indicate whether an index (e.g., the ClusterIndex) has been changed (e.g., change history) compared to previous data. TRUE may be displayed when the index has been changed, and FALSE may be displayed when the index has not been changed. For example, since an index of data with Datetimes “2021 May 26” is (7, 7) and an index of data with Datetimes “2021 Jul. 3” has been changed into (7, 6), the ClusterChange of the Datetimes “2021 Jul. 3” may be displayed as TRUE.

An electronic device may extract, from the time series data, data with an index changed compared to previous data. The electronic device may determine movement rate information representing a rate of movement from each index to each of adjacent indices, based on the extracted data.

In the case in which only data obtained through actual measurement (e.g., data with Datasource “real”) is used, when an interval between dates (e.g., an interval of Datetimes) is large, it may not be possible to determine a timepoint at which the index has been changed. For example, when the user measures body indicators after one month and the indices have been changed compared to previous data, it may not be possible to determine an exact timepoint at which the indices have been changed. When the timepoint at which the indices have been changed is not determined, there may be errors in the movement rate information. The electronic device may fill in gaps in the data through interpolation. Time series data generated through interpolation is described below with reference to FIG. 11.

The electronic device may, based on the extracted data, determine movement directions of indices of each of a plurality of users for a determined period of time (e.g., one year). The movement directions of the indices are described below. The movement directions of the indices may be used as a reference material.

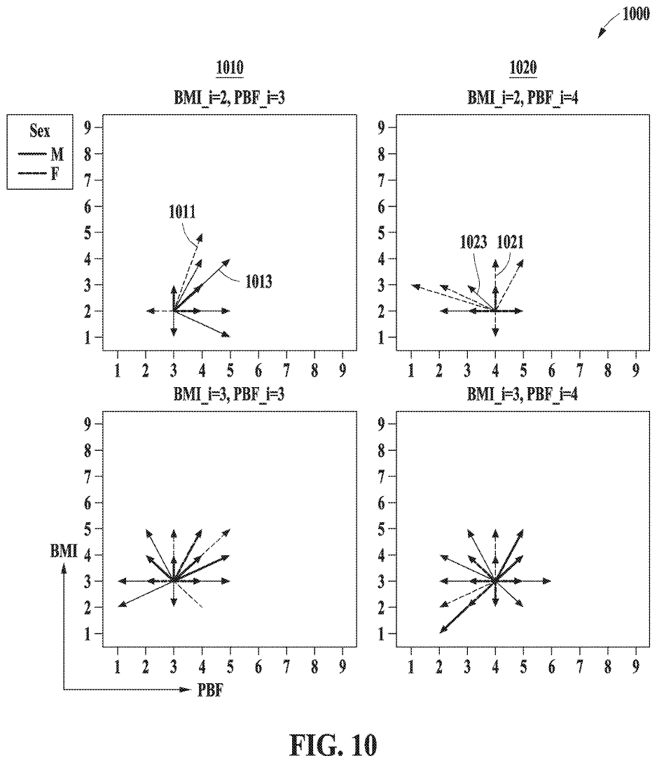

FIG. 10 is a diagram illustrating a movement direction according to an embodiment.

Referring to FIG. 10, a graph 1000 is shown in which movement directions of indices of users with a BMI_i (e.g., BMI_index) of 2 or 3 or a PBF_i (e.g., PBF_index) of 3 or 4 for a determined period of time are illustrated. It should be understood that, although there may be a total of 81 graphs since a 2D space with BMI and PBF as axes is divided into 9 percentile segments, only 4 thereof are illustrated for ease of description. In addition, while FIG. 10 only distinguishes between genders, it may be obvious to one of ordinary skill in the art that additional graphs illustrating movement directions of indices distinguished by age groups may be created.

A graph 1010 illustrates movement directions of indices of users with a BMI_i of 2 and a PBF_i of 3 (e.g., an index (2, 3)) for a determined period of time. For example, some of women with the BMI_i of 2 and the PBF_i of 3 may have moved along an arrow 1011. For example, some of the women included in the index (2, 3) may have moved to (5, 4) along the arrow 1011. For example, some of men with the BMI_i of 2 and the PBF_i of 3 may have moved along an arrow 1013. For example, some of the men included in the index (2, 3) may have moved to an index (4, 5) along the arrow 1013. The boldness (e.g., thickness) of an arrow may be related to a magnitude of the movement rate. As an arrow becomes bolder, more users may have moved in the corresponding direction.

A graph 1020 illustrates movement directions of indices of users with a BMI_i of 2 and a PBF_i of 4 for a determined period of time. For example, some of women with the BMI_i of 2 and the PBF_i of 4 may have moved along an arrow 1021. For example, some of the women included in an index (2, 4) may have moved to (4, 4) along the arrow 1021. For example, some of men with the BMI_i of 2 and the PBF_i of 4 may have moved along an arrow 1023. For example, some of the men included in the index (2, 4) may have moved to an index (3, 3). The boldness of an arrow may be related to a magnitude of the movement rate. As an arrow becomes bolder, more users may have moved in the corresponding direction.

FIG. 11 is a diagram illustrating interpolated time series data according to an embodiment.

Referring to FIG. 11, a portion of time series data is illustrated, which is obtained by performing interpolation on time series data of body indicators pre-obtained from a plurality of users. For ease of description, the data illustrated in FIG. 11 is referred to as interpolated time series data 1100, although being a portion of interpolated data. The interpolated time series data 1100 may include the same plurality of fields as the time series data 900 of FIG. 9 and thus, detailed description is omitted herein.

In the interpolated time series data 1100, data obtained through actual measurement may be indicated as “real” and interpolated data may be indicated as “fake” in the DataSource field.

An electronic device may perform interpolation when a difference between a measurement date of particular data and a measurement date (e.g., Datetimes) of previous data in the time series data exceeds a threshold time (e.g., 1 day). The threshold time may vary depending on settings. For example, the threshold time may be determined as 1 day, 3 days, or 1 week. For example, the electronic device may determine that data with Datetimes “2021 Jul. 3” is one week apart from previous data, data with Datetimes “2021 Jun. 26.” For example, since one week exceeds the threshold time (e.g., 1 day), the electronic device may perform interpolation.

The electronic device may perform data interpolation using various interpolation methods, such as linear interpolation, polynomial interpolation, Lagrange interpolation, and Newton interpolation. For example, the electronic device may perform data interpolation using linear interpolation. The electronic device may estimate a median value of measurement dates of data having adjacent indices as a point at which the indices have changed. The adjacent indices may refer to indices having a difference of 1 between BMI_i's and/or PBF_i's.

The electronic device may perform interpolation on a daily basis. The electronic device may estimate a point at which an index is changed through interpolation. Although not shown in the interpolated time series data 1100, ClusterChange of the interpolated data may also be determined as TRUE.

The electronic device may extract, from the interpolated time series data 1100, data with an index changed compared to previous data. The electronic device may determine movement rate information representing a rate of movement from each index to each of adjacent indices, based on the extracted data.

The movement rate information is described below.

FIG. 12 is a diagram illustrating movement rate information according to an embodiment.

For ease of description, only movement rate information of cases with a BMI i (e.g., BMI_index) of 4 or 3 or a PBF_i (e.g., PBF_index) of 3 or 4 is illustrated in FIG. 12. It should be understood that, although there may be a total of 81 pieces of movement rate information since a 2D space with BMI and PBF as axes is divided into 9 percentile segments, only 4 thereof are illustrated for ease of description.

The electronic device may determine movement rate information representing a rate of movement from each index to each of adjacent indices, based on data extracted from time series data. An index may move to an adjacent index of the corresponding index. For example, an index may move to at least one of up, down, left, right, upper left, upper right, lower left, and lower right. For example, movement rate information 1210 may represent that indices of users with the BMI_i of 4 and the PBF_i of 3 have moved to up, down, left, right, and upper left.

The boldness of an arrow may be related to a magnitude of the movement rate. As an arrow becomes bolder, more users may have moved in the corresponding direction. For example, since an arrow 1220 is the boldest in the movement rate information 1210, it may indicate that the users with the BMI_i of 4 and the PBF_i of 3 have moved the most to an index of the BMI_i of 4 and the PBF_i of 2. For example, it may indicate that 30%, the largest proportion, of the users with the BMI_i of 4 and the PBF_i of 3 has moved along the arrow 1220.

The electronic device may generate a histogram based on the movement rate information. The electronic device may generate a cumulative distribution function based on the histogram. This is described below with reference to FIG. 13.

FIG. 13 is a diagram illustrating a histogram and cumulative distribution function based on movement rate information, according to an embodiment.

An electronic device may, based on movement rate information, generate a histogram 1300 with a movement rate and a number of pieces of data included in the movement rate information as axes.

In the histogram 1300, a path may represent an arrow. A total number of paths may indicate a total number of arrows included in the movement rate information. As each of 81 indices may have a maximum of 8 movement directions, the total number of paths may be a maximum of 648 (e.g., 81×8).

In the histogram 1300, a movement rate of each path for the entire indices may indicate a rate represented by an arrow in the movement rate information. In the histogram 1300, the number of pieces of data corresponding to the movement rate may indicate the number of arrows in the movement rate information.

For example, the arrow 1220 of the movement rate information 1210 of FIG. 12 represents 30% and may thus be included in 0.3, which may be the movement rate of each path for the entire indices.

The electronic device may generate a cumulative distribution function 1310 based on the histogram 1300. The cumulative distribution function 1310 may represent a cumulative rate of a path according to a movement rate of each path for the entire indices. For example, referring to the cumulative distribution function 1310, the rate of paths corresponding to the movement rate of each path of 0% to 0.05% may be 19% of the total.

The electronic device may divide the cumulative distribution function 1310 into a plurality of segments. For example, the electronic device may divide the cumulative distribution function 1310 into a first segment 1340, a second segment 1350, and a third segment 1360. However, embodiments are not limited thereto. The first segment 1340 may be a segment corresponding to a difficulty level “high,” the second segment 1350 may be a segment corresponding to a difficulty level “medium,” and the third segment 1360 may be a segment corresponding to a difficulty level “low.”

For example, referring to the cumulative distribution function 1310, a cumulative rate of a path having a movement rate of 0.02 may be assumed to be 0.05 (e.g., top 5%). Since the cumulative rate of the path having the movement rate of 0.02 may be 0.05, which may rarely happen, the path having the movement rate of 0.02 may correspond to the difficulty level “high.” For example, referring to the cumulative distribution function 1310, a cumulative rate of a path having a movement rate of 0.25 may be assumed to be 0.9 (e.g., top 90% or lower 10%). The path having the movement rate of 0.25 may have the cumulative rate of 0.9 and may thus correspond to the difficulty level “low.”

The electronic device may divide the cumulative distribution function 1310 into a plurality of segments by generating one or more boundary lines 1320 and 1330. The electronic device may generate the one or more boundary lines 1320 and 1330 based on at least one of an inflection point or a point at which a change rate of the cumulative distribution function 1310 exceeds a threshold rate. The electronic device may generate the plurality of segments, which may represent different difficulty levels, based on the one or more boundary lines 1320 and 1330.

The electronic device may determine, based on the cumulative distribution function 1310, a difficulty level expected to be taken for a body indicator of a target user to move from a starting cluster to a most adjacent cluster on a target route.

Hereinafter, determination of expected time is described.

FIGS. 14 and 15 are diagrams illustrating a determination of expected time, according to an embodiment.

Time information, which may represent time taken per unit distance consumed in moving from each index to each of adjacent indices, may be determined based on extracted data. An electronic device may determine the time information representing the time taken per unit distance by dividing the time consumed in moving from each index to each of the adjacent indices in a coordinate space, which uses body indicators as coordinates, by a movement distance of the index. Hereinafter, a method of determining the time consumed per unit distance is described.

Even users having a same index may be at different actual positions in that index. For example, when a user C and a user D are both included in an index with the BMI_index of 7 and the PBF_index of 3, an actual position indicated by a body indicator of the user C may be a point 1410, and an actual position indicated by a body indicator of the user D may be a point 1450.

It may be assumed that the user C and the user D have both moved from the index with the BMI_index of 7 and the PBF_index of 3 to an index with the BMI_index of 7 and the PBF_index of 2. For example, it may be assumed that the user C has moved from the point 1410 to a point 1420 and that the user D has moved from the point 1450 to a point 1460.

An actual movement distance of the body indicator of the user C may be a distance 1430. An actual movement distance of the body indicator of the user D may be a distance 1470. For example, the distance 1430 may be shorter than the distance 1470. The distance of actual movement may be referred to as “Euclidean distance.”

According to an embodiment, the electronic device may apply different weights for each body indicator when determining movement distances of body indicators. The electronic device may apply a greater weight to a body indicator that is relatively more difficult to change. For example, the electronic device may apply a greater weight to the BMI_index. For example, the electronic device may apply a weight of 0.7 to the BMI_index and apply a weight of 0.3 to the PBF_index. By applying a greater weight to the BMI_index, a distance may be determined to be longer for a case in which a decrease in a BMI is greater, even when the degree of a change in the body indicators is similar.

The electronic device may determine the time information by dividing the time consumed by a user in changing an index by a movement distance of a body indicator of the user to normalize into a time taken per unit distance. The movement distance of the body indicator may be a distance to which a Euclidean distance or a weight is applied. For example, it may be assumed that the user C took 50 days to move the distance 1430 and that the user D took 50 days to move the distance 1470. When normalizing the time into the time taken per unit distance by dividing the time consumed by the user in changing an index by the movement distance of the body indicator of the user, the time taken per unit distance of the user C may be greater.

Since the extracted time series data may include a measurement date, PBF, BMI, BMI_index, PBF_index, and ClusterChange, the electronic device may determine the time information representing the time taken per unit distance by dividing the time consumed in moving from each index to each of the adjacent indices in a coordinate space, which uses body indicators as coordinates, by the movement distance. The time information may be the time taken per unit distance represented as a histogram.

Referring to FIG. 15, time information 1500 is illustrated, which represents time taken per unit distance of users that moved from an index (5, 3) to the left (e.g., (5, 2)). For ease of description, only the time information 1500 representing the time taken per unit distance of the users that moved from the index (5, 3) to the left (e.g., (5, 2)) is shown in FIG. 15. It should be understood that, however, more time information may be determined, since a 2D space with BMI and PBF as axes is divided into 9 percentile segments and thus, movements may be made from a total of 81 indices in 8 directions (e.g., up, down, left, right, upper left, upper right, lower left, and lower right).

The electronic device may, based on the time information 1500, determine expected time to be taken for a body indicator of a target user to move from a starting cluster to a most adjacent cluster on a target route.

The electronic device may determine the expected time using any one of a mode, a median, a mean, and 2 sigma. The electronic device may generate a baseline 1510 based on any one of the mode, the median, the mean, and the 2 sigma. The electronic device may determine the time taken per unit distance intersecting the baseline 1510 as the expected time.

The electronic device may determine the expected time by selecting any one of the mode, the median, the mean, and the 2 sigma according to a disposition of a target user. The disposition of a target user may be input through settings. For example, a target user with a challenging disposition may desire rapid achievement, and accordingly, the electronic device may determine the expected time based on the 2 sigma. For example, a target user with a stable disposition may desire assured achievement, and accordingly, the electronic device may determine the expected time based on the mean.

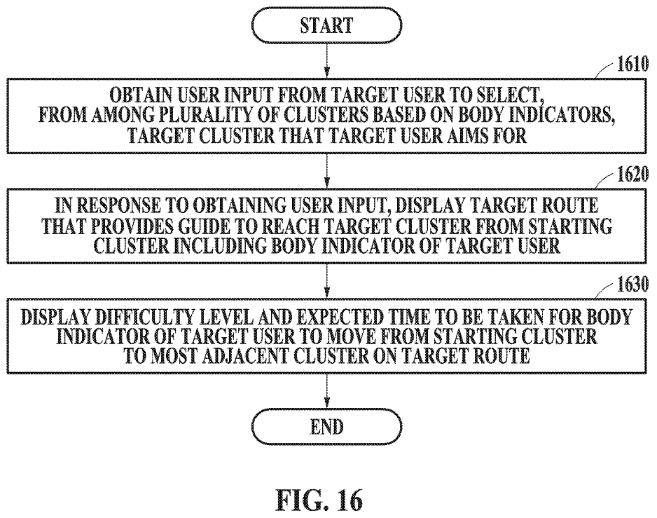

FIG. 16 is a flowchart illustrating an operation of a user terminal, according to an embodiment.

In the following embodiments, operations may be performed sequentially but not necessarily. For example, the order of the operations may change, and at least two of the operations may be performed in parallel. In addition, some of the operations may be omitted depending on the embodiment. Operations 1610 to 1630 may be performed by at least one component (e.g., a processor) of the user terminal. According to an embodiment, instructions stored in memory by at least one processor may be executed individually or collectively and may cause the user terminal to perform the following operations.

In operation 1610, the user terminal may obtain a user input from a target user to select, from among a plurality of clusters based on body indicators, a target cluster that the target user aims for.

In operation 1620, the user terminal may, in response to obtaining the user input, display a target route that provides a guide to reach the target cluster from a starting cluster including a body indicator of the target user.

In operation 1630, the user terminal may display a difficulty level and expected time to be taken for the body indicator of the target user to move from the starting cluster to a most adjacent cluster on the target route.

An electronic device communicating with the user terminal may, in response to receiving the user input from the user terminal, determine the target route, the time, and the difficulty level based on time series data of body indicators pre-obtained from a plurality of users. The electronic device may provide the target route, the expected time, and the difficulty level to the user terminal.

Operations 1610 to 1650 are described in detail with reference to FIGS. 1 to 15. Thus, a detailed description of operations 1610 to 1650 is omitted herein.

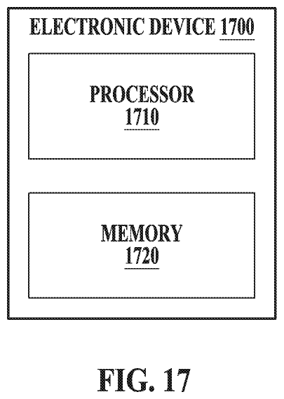

FIG. 17 is a diagram illustrating an electronic device according to an embodiment.

According to an embodiment, an electronic device 1700 may guide goal setting and goal achievement based on body indicators. The electronic device 1700 may include, but not be limited to, various computing devices such as a mobile phone, a smartphone, a tablet, an electronic book device, a laptop, a personal computer, a desktop computer, a workstation, or a server, various wearable devices such as a smart watch, smart glasses, or a head-mounted display (HMD), various home appliances such as a smart speaker, a smart television (TV), or a smart refrigerator, a smart vehicle, a smart kiosk, an Internet of things (IoT) device, a wearable assistive device (WAD), a drone, a robot, or a body indicator measuring device.

The electronic device 1700 may include a processor 1710 and a memory 1720. The processor 1710 may perform overall functions for controlling the electronic device 1700. The processor 1710 may generally control the electronic device 1700 by executing programs and/or instructions stored in the memory 1720. The processor 1710 may be implemented as a central processing unit (CPU), a graphics processing unit (GPU), an access point (AP), and the like. However, embodiments are not limited thereto. The number of the processor 1710 may be one or more. For example, the processor 1710 may have a structure of a multi-core processor such as a dual core, a quad core, or a hexa core. The processor 1710 may control operations of the electronic device 1700 by executing the instructions stored in the memory 1720. For example, the processor 1710 may correspond to a plurality of processors that collectively performs a plurality of operations by dividing them among processors.

The memory 1720 may be hardware for storing data processed and data to be processed in the electronic device 1700. In addition, the memory 1720 may store an application, a driver, and the like to be driven by the electronic device 1700. Furthermore, the memory 1720 may include the instructions. The memory 1720 may include one or more memories. The instructions stored in the memory 1720 may be stored in one memory. The instructions stored in the memory 1720 may be divided and stored in a plurality of memories. The memory 1720 may include a volatile memory, such as dynamic random-access memory (DRAM), and/or a non-volatile memory.

The electronic device 1700 may select, from among a plurality of clusters based on body indicators, a target cluster that the target user aims for, based on a user input. The electronic device 1700 may determine, based on time series data of body indicators pre-obtained from a plurality of users, a target route to guide the target user from a starting cluster including a body indicator of the target user to the target cluster. The electronic device 1700 may determine, based on the time series data, a difficulty level and expected time to be taken for the body indicator of the target user to move from the starting cluster to a most adjacent cluster on the target route. The electronic device 1700 may provide the target route, the time, and the difficulty level to the target user.

The electronic device 1700 may determine a plurality of candidate paths based on the starting cluster and the target cluster, from among a plurality of paths predetermined based on indices representing position information of the body indicators. The electronic device 1700 may determine a target path among the plurality of candidate paths and determine the target route based on the target path.

The electronic device 1700 may determine a plurality of candidate paths starting from an index representing the body indicator of the target user and ending at any index included in the target cluster.

The electronic device 1700 may extract, from time series data, data with an index changed compared to previous data, wherein the index may represent statistical position information of the body indicator. The electronic device 1700 may determine movement rate information representing a rate of movement from each index to each of adjacent indices, based on the extracted data. The electronic device 1700 may determine a difficulty level based on the movement rate information.

The electronic device 1700 may, based on the movement rate information, generate a histogram with a rate and a number of pieces of data included in the movement rate information as axes. The electronic device 1700 may determine a cumulative distribution function based on the histogram. The electronic device 1700 may determine a difficulty level based on the cumulative distribution function.

The electronic device 1700 may extract, from time series data, data with an index changed compared to previous data, wherein the index may represent statistical position information of the body indicator. The electronic device 1700 may determine time information representing time taken per unit distance consumed in moving from each index to each of adjacent indices, based on the extracted data. The electronic device 1700 may determine expected time based on the time information.

The electronic device 1700 may determine the time information representing the time taken per unit distance by dividing the time consumed in moving from each index to each of the adjacent indices in a coordinate space, which uses body indicators as coordinates, by a movement distance of the index.

A body indicator may include a BMI and a PBF.

Based on the user input, the electronic device 1700 may select, as the target cluster, any one of top two or more clusters with highest scores based on the body indicators.

Based on the user input, the electronic device 1700 may select, as the target cluster, any one of clusters with scores based on the body indicators greater than or equal to an average.

Based on the user input, the electronic device 1700 may select, as the target cluster, any one of clusters determined based on a period set by the target user.

It is obvious to one of ordinary skill in the art that the description of the operations provided above with reference to FIGS. 1 to 16 may apply to the operations of the electronic device 1700 described above.

The method according to embodiments may be written in a computer-executable program and may be implemented as various recording media such as magnetic storage media, optical reading media, or digital storage media.

Various techniques described herein may be implemented in digital electronic circuitry, computer hardware, firmware, software, or combinations thereof. Implementations may implemented as a computer program product, i.e., a computer program tangibly embodied in an information carrier, e.g., in a machine-readable storage device (computer-readable medium), for processing by, or to control an operation of, a data processing apparatus, e.g., a programmable processor, a computer, or multiple computers. A computer program, such as the computer program(s) described above, may be written in any form of a programming language, including compiled or interpreted languages, and may be deployed in any form, including as a stand-alone program or as a module, a component, a subroutine, or other units suitable for use in a computing environment. A computer program may be deployed to be processed on one computer or multiple computers at one site or distributed across multiple sites and interconnected by a communication network.

Processors suitable for processing of a computer program include, by way of example, both general and special-purpose microprocessors, and any one or more processors of any type of digital computer. Generally, a processor will receive instructions and data from read-only memory (ROM) or random-access memory (RAM), or both. Elements of a computer may include at least one processor for executing instructions and one or more memory devices for storing instructions and data. Generally, a computer may also include, or be operatively coupled to receive data from or transfer data to, or both, one or more mass storage devices for storing data, e.g., magnetic, magneto-optical disks, or optical disks. Examples of information carriers suitable for embodying computer program instructions and data include semiconductor memory devices, for example, magnetic media such as hard disks, floppy disks, and magnetic tape, optical media such as compact disc ROM (CD-ROM) or a digital versatile disc (DVD), magneto-optical media such as floptical disks, ROM, RAM, flash memory, erasable programmable ROM (EPROM), or electrically erasable programmable ROM (EEPROM). The processor and the memory may be supplemented by, or incorporated in, special-purpose logic circuitry.

In addition, non-transitory computer-readable media may be any available media that may be accessed by a computer and may include both computer storage media and transmission media.

Although the present specification includes details of a plurality of specific embodiments, the details should not be construed as limiting any invention or a scope that can be claimed, but rather should be construed as being descriptions of features that may be peculiar to specific embodiments of specific inventions. Specific features described in the present specification in the context of individual embodiments may be combined and implemented in a single embodiment. On the contrary, various features described in the context of a single embodiment may be implemented in a plurality of embodiments individually or in any appropriate sub-combination. Moreover, although features may be described above as acting in specific combinations and even initially claimed as such, one or more features from a claimed combination can in some cases be excised from the combination, and the claimed combination may be changed to a sub-combination or a modification of a sub-combination.

Likewise, although operations are depicted in a predetermined order in the drawings, it should not be construed that the operations need to be performed sequentially or in the predetermined order, which is illustrated to obtain a desirable result, or that all of the shown operations need to be performed. In specific cases, multi-tasking and parallel processing may be advantageous. In addition, it should not be understood that the separation of various device components of the aforementioned embodiments is required in all types of embodiments, and it should be understood that the described program components and devices are generally integrated as a single software product or packaged into a multiple-software product.

The embodiments disclosed in the present specification and the drawings are intended merely to present specific examples in order to aid in understanding of the present disclosure, but are not intended to limit the scope of the present disclosure. It will be apparent to one of ordinary skill in the art that, in addition to the disclosed embodiments, various other examples modified based on the technical spirit of the present disclosure may be implemented.

1. An operating method of an electronic device, the operating method comprising:
selecting, from among a plurality of clusters based on body indicators, a target cluster that a target user aims for, based on a user input;
determining, based on time series data of body indicators pre-obtained from a plurality of users, a target route that guides the target user from a starting cluster comprising a body indicator of the target user to the target cluster;
determining, based on the time series data, a difficulty level and expected time to be taken for the body indicator of the target user to move from the starting cluster to a most adjacent cluster on the target route; and
providing the target route, the time, and the difficulty level to the target user.

2. The operating method of claim 1, wherein the determining of the target route comprises:
determining, from among a plurality of paths, a plurality of candidate paths based on the starting cluster and the target cluster, wherein the plurality of paths is predetermined based on an index representing position information of the body indicators; and
determining a target path among the plurality of candidate paths and determining the target route based on the target path.

3. The operating method of claim 2, wherein the determining of the plurality of candidate paths comprises:
determining a plurality of candidate paths starting from an index representing the body indicator of the target user and ending at any index included in the target cluster.

4. The operating method claim 1, wherein the determining of the expected time and the difficulty level comprises:
extracting, from the time series data, data with an index changed compared to previous data, wherein the index represents statistical position information of the body indicator;
determining movement rate information representing a rate of movement from each index to each of adjacent indices, based on the extracted data; and
determining the difficulty level based on the movement rate information.

5. The operating method of claim 4, wherein the determining of the difficulty level based on the movement rate information comprises:
generating, based on the movement rate information, a histogram with a rate and a number of pieces of data included in the movement rate information as axes;
determining a cumulative distribution function based on the histogram; and
determining the difficulty level based on the cumulative distribution function.

6. The operating method of claim 1, wherein the determining of the expected time and the difficulty level comprises:
extracting, from the time series data, data with an index changed compared to previous data, wherein the index represents statistical position information of the body indicator;
determining, based on the extracted data, time information representing time taken per unit distance consumed in moving from each index to each of adjacent indices; and
determining the expected time based on the time information.

7. The operating method of claim 6, wherein the determining of the time information comprises:
determining the time information representing the time information representing the time taken per unit distance by dividing time consumed in moving from each index to each of the adjacent indices in a coordinate space, which uses the body indicators as coordinates, by a movement distance of the index.

8. The operating method of claim 1, wherein the body indicators comprise:
a body mass index (BMI) and a percentage body fat (PBF).

9. The operating method of claim 1, wherein the selecting of the target cluster comprises:
selecting, based on the user input, any one of top two or more clusters with the highest scores based on the body indicators as the target cluster.

10. The operating method of claim 1, wherein the selecting of the target cluster comprises:
selecting, based on the user input, any one of clusters with scores based on the body indicators greater than or equal to an average as the target cluster.

11. The operating method of claim 1, wherein the selecting of the target cluster comprises:
selecting, based on the user input, any one of clusters determined based on a period set by the target user as the target cluster.

12. An operating method of a user terminal, the operating method comprising:
obtaining a user input from a target user to select, from among a plurality of clusters based on body indicators, a target cluster that the target user aims for;
displaying, in response to obtaining the user input, a target route that provides a guide to reach the target cluster from a starting cluster comprising a body indicator of the target user; and
displaying a difficulty level and expected time to be taken for the body indicator of the target user to move from the starting cluster to a most adjacent cluster on the target route,
wherein an electronic device communicating with the user terminal is configured to:
in response to receiving the user input from the user terminal, determine the target route, the time, and the difficulty level based on time series data of body indicators pre-obtained from a plurality of users, and provide the target route, the expected time, and the difficulty level to the user terminal.

13. An electronic device comprising:
at least one processor comprising processing circuitry; and
memory storing instructions that, when executed by the at least one processor individually or collectively, cause the electronic device to:
select, from among a plurality of clusters based on body indicators, a target cluster that a target user aims for, based on a user input;
determine, based on time series data of body indicators pre-obtained from a plurality of users, a target route that guides the target user from a starting cluster comprising a body indicator of the target user to the target cluster;
determine, based on the time series data, a difficulty level and expected time to be taken for the body indicator of the target user to move from the starting cluster to a most adjacent cluster on the target route; and
provide the target route, the time, and the difficulty level to the target user.

14. The electronic device of claim 13, wherein the instructions, when executed by the at least one processor individually or collectively, cause the electronic device to:
determine, from among a plurality of paths, a plurality of candidate paths based on the starting cluster and the target cluster, wherein the plurality of paths is predetermined based on an index representing position information of the body indicators; and
determine a target path among the plurality of candidate paths and determine the target route based on the target path.

15. The electronic device of claim 14, wherein the instructions, when executed by the at least one processor individually or collectively, cause the electronic device to:
determine a plurality of candidate paths starting from an index representing the body indicator of the target user and ending at any index included in the target cluster.

16. The electronic device of claim 13, wherein the instructions, when executed by the at least one processor individually or collectively, cause the electronic device to:
extract, from the time series data, data with an index changed compared to previous data, wherein the index represents statistical position information of the body indicator;
determine movement rate information representing a rate of movement from each index to each of adjacent indices, based on the extracted data; and
determine the difficulty level based on the movement rate information.

17. The electronic device of claim 16, wherein the instructions, when executed by the at least one processor individually or collectively, cause the electronic device to:
generate, based on the movement rate information, a histogram with a rate and a number of pieces of data included in the movement rate information as axes;
determine a cumulative distribution function based on the histogram; and
determine the difficulty level based on the cumulative distribution function.

18. The electronic device of claim 13, wherein the instructions, when executed by the at least one processor individually or collectively, cause the electronic device to:
extract, from the time series data, data with an index changed compared to previous data, wherein the index represents statistical position information of the body indicator;
determine, based on the extracted data, time information representing time taken per unit distance consumed in moving from each index to each of adjacent indices; and
determine the expected time based on the time information.

19. The electronic device of claim 18, wherein the instructions, when executed by the at least one processor individually or collectively, cause the electronic device to:
determine the time information representing the time information representing the time taken per unit distance by dividing time consumed in moving from each index to each of the adjacent indices in a coordinate space, which uses the body indicators as coordinates, by a movement distance of the index.

20. The electronic device of claim 13, wherein the body indicators comprise:
a body mass index (BMI) and a percentage body fat (PBF).
